(19) United States
(12) Patent Application Publication
(10) Pub. No.: US 2022/0249065 A1
(43) Pub. Date: Aug. 11, 2022

(54) ULTRASOUND DIAGNOSTIC APPARATUS, SETTING CONTROL METHOD, AND SETTING CONTROL PROGRAM

(71) Applicant: Konica Minolta, Inc., Tokyo (JP)

(72) Inventors: Ryota TORII, Tokyo (JP); Tomohito SAKAI, Yokohama-shi (JP)

(21) Appl. No.: 17/649,876

(22) Filed: Feb. 3, 2022

(30) Foreign Application Priority Data
Feb. 9, 2021 (JP) 2021-019069

(51) Int. Cl. A61B 8/00

(52) U.S. Cl. CPC A61B 8/54; A61B 8/461; A61B 8/4488

(57) ABSTRACT

An ultrasound diagnostic apparatus for generating an ultrasound image of a subject by driving an ultrasound probe in which a plurality of transducers are arranged in an array, includes a hardware processor that sets a region of interest of the ultrasound image and sets a scanning method of the ultrasound probe in accordance with a position of the set region of interest.

The entire disclosure of Japanese patent Application No. 2021-019069, filed on Feb. 9, 2021, is incorporated herein by reference in its entirety.

### BACKGROUND

### Technological Field

The present invention relates to an ultrasound diagnostic apparatus, a setting control method, and a setting control program.

### Description of the Related Art

Conventionally, as one of medical image diagnostic apparatuses, there is known an ultrasound diagnostic apparatus that visualizes a shape, a property, or dynamics inside a subject as an ultrasound image, by transmitting an ultrasound wave toward the subject, receiving a reflected wave, and performing predetermined signal processing on a reception signal. Since the ultrasound diagnostic apparatus can acquire an ultrasound image by a simple operation of applying an ultrasound probe to a body surface or inserting the ultrasound probe into a body, the ultrasound diagnostic apparatus is safe, and a burden on a subject is small.

In an ultrasound diagnostic apparatus of an electronic scanning method, phased array technology is utilized that can control a beam direction of ultrasound waves and a shape of the ultrasound waves, for example, by using an ultrasound probe (so-called array probe) in which a plurality of transducers are arranged in an array and electronically changing a drive timing of each transducer. In the electronic scanning method, a diagnosis target can be scanned along an arrangement direction (hereinafter, referred to as a “scanning direction”) of the transducers by driving a transducer group including a plurality of continuous transducers while sequentially shifting in the arrangement direction of the transducers.

Further, as an example of an electronic scanning method, trapezoidal scanning (trapezoid scanning) in which a beam direction of an ultrasound wave is changed has been put into practical use. By performing such trapezoidal scanning, it is possible to perform scanning in a range that is wider than the entire width of the plurality of transducers, which makes it possible to expand a diagnosis region of the ultrasound diagnostic apparatus.

Meanwhile, there is a case where a partial region of an ultrasound image generated by the ultrasound diagnostic apparatus is set as a region of interest. Considering that a portion of the region of interest is, for example, a portion where a user desires detailed observation in the ultrasound image, it is desirable that the portion of the region of interest is an image with high resolution.

For example, JP H6-217981 A achieves improvement of image quality of a region of interest by performing control to increase the number of scanning lines in the region of interest. In addition, in JP 2011-239906 A, blurring when an ultrasound image is enlarged is suppressed by increasing the number of sampling points on each scanning line, increasing a density of the scanning lines, and the like, when the ultrasound image is enlarged.

However, in a configuration in which the number of acoustic lines (scanning lines) in a region is increased as in JP H6-217981 A and JP 2011-239906 A, a range of a settable region of interest is likely to be limited. The region of interest may be set at various locations depending on a subject. In particular, when the above-described trapezoidal scanning is performed, an inter-acoustic line angle between two adjacent ultrasound waves is wider than that when normal scanning is performed, and thus, a setting range of a position of a region of interest is also wider. Therefore, with the configurations described in JP H6-217981 and JP 2011-239906 A, there has been a possibility that an image related to a region of interest is unable to be generated depending on a position of the region of interest.

### SUMMARY

An object of the present invention is to provide an ultrasound diagnostic apparatus, a setting control method, and a setting control program capable of handling various region of interest positions.

To achieve the abovementioned object, according to an aspect of the present invention, an ultrasound diagnostic apparatus for generating an ultrasound image of a subject by driving an ultrasound probe in which a plurality of transducers are arranged in an array, reflecting one aspect of the present invention comprises a hardware processor that sets a region of interest of the ultrasound image, and sets a scanning method of the ultrasound probe in accordance with a position of the set region of interest.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a view illustrating an appearance of an ultrasound diagnostic apparatus according to an embodiment of the present invention;

FIG. 2 is a view illustrating an example of a scannable range of an ultrasound probe;

FIG. 3 is a block diagram illustrating a main part of a control system of the ultrasound diagnostic apparatus;

FIG. 4 is a view for explaining an acoustic line angle;

FIG. 5 is a view illustrating an example of a scannable range of trapezoid scanning;

FIG. 6 is a view for explaining that an inter-acoustic line angle differs depending on a scanning method;

FIG. 7 is a flowchart illustrating an operation example of setting control of a scanning method in a controller;

FIG. 8 is a graph illustrating an example of a function for determining a value of a deflection angle variation coefficient;

FIG. 9 is a view for explaining that an inter-acoustic line angle differs depending on a value of the deflection angle variation coefficient; and

FIG. 10 is a graph illustrating an example of a function for determining a value of the deflection angle variation coefficient.

### DETAILED DESCRIPTION OF EMBODIMENTS

Hereinafter, one or more embodiments of the present invention will be described with reference to the drawings. However, the scope of the invention is not limited to the disclosed embodiments.

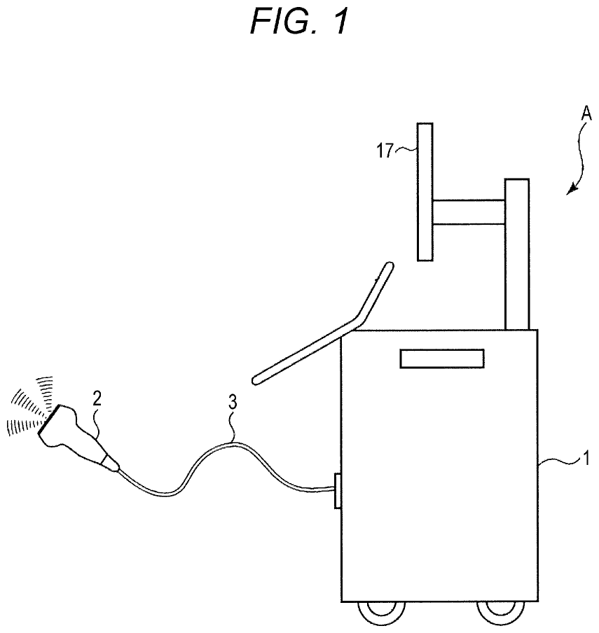

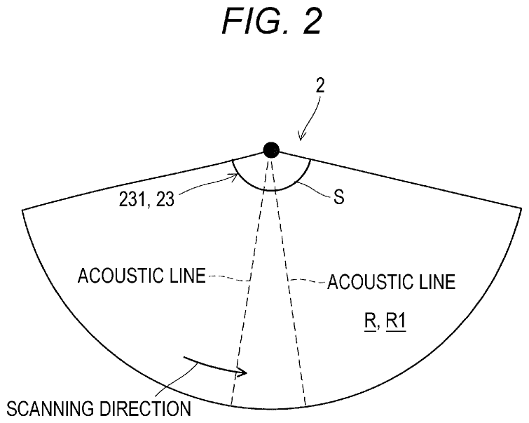

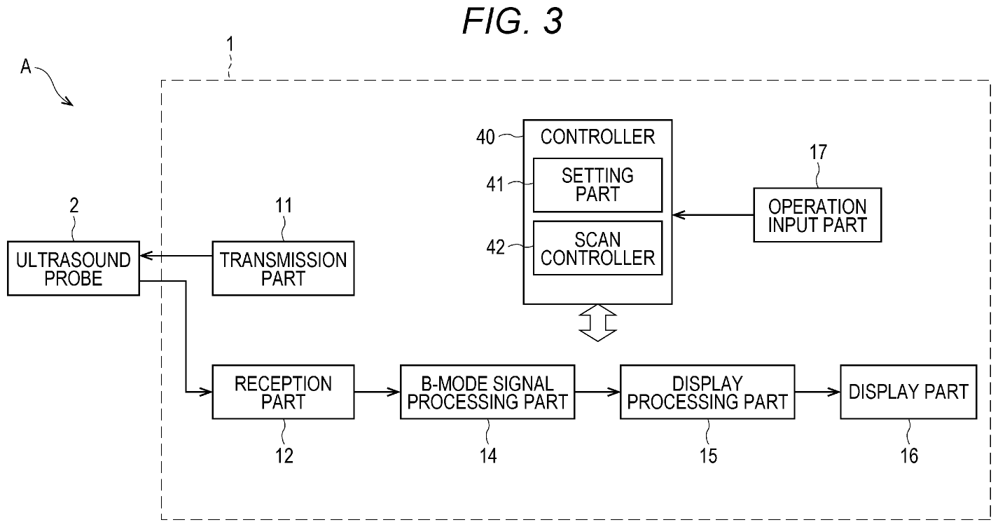

FIG. 1 is a view illustrating an appearance of an ultrasound diagnostic apparatus A according to an embodiment of the present invention. FIG. 2 is a view illustrating an example of a scannable range of an ultrasound probe 2. FIG. 3 is a block diagram illustrating a main part of a control system of the ultrasound diagnostic apparatus A.

As illustrated in FIG. 1, the ultrasound diagnostic apparatus A includes an ultrasound diagnostic apparatus main body 1 and the ultrasound probe 2. The ultrasound diagnostic apparatus main body 1 and the ultrasound probe 2 are connected via a cable 3. The ultrasound probe 2 may be connected to the ultrasound diagnostic apparatus main body 1 via wireless communication.

The ultrasound diagnostic apparatus A is used to visualize a shape, a property, or dynamics inside a subject as an ultrasound image and perform image diagnosis. The ultrasound diagnostic apparatus A has a B mode for displaying a B-mode image alone, as a display mode. The ultrasound diagnostic apparatus A may have a CFM mode in which a color flow mapping (CFM) image obtained by a color Doppler method is superimposed and displayed on a B-mode image.

The ultrasound probe 2 transmits an ultrasound wave to the subject, receives an ultrasound echo reflected by the subject, converts the ultrasound echo into a reception signal, and transmits the reception signal to the ultrasound diagnostic apparatus main body 1. The ultrasound probe 2 is a probe compatible with an electronic scanning method, and for example, a linear probe, a convex probe, or a sector probe can be applied. In the present embodiment, a case will be described in which a probe (for example, a convex probe) capable of handling a wider diagnosis region is applied as the ultrasound probe 2.

As illustrated in FIG. 2, the ultrasound probe 2 includes a transducer array 23. The transducer array 23 includes a plurality of transducers 231 arranged in a scanning direction.

The plurality of transducers 231 are arranged such that transducer surfaces S are arranged on an arc. Therefore, the scanning direction is a direction along the arc formed by the transducer surfaces S (for example, a counterclockwise direction in the illustration). Such a transducer array 23 causes a diagnosis region R in the ultrasound diagnostic apparatus A to be a fan-shaped region. In FIG. 2, each of the plurality of transducers 231 is indicated by a line connecting the plurality of transducers with a curve.

According to the ultrasound probe 2, an ultrasound wave can converge in the scanning direction (so-called electronic focus) by sequentially switching the transducer 231 to be driven in the scanning direction.

Note that acoustic lines corresponding to the number of transducers 231 pass through the diagnosis region R, but in FIG. 2, two acoustic lines alone of ultrasound waves are illustrated in consideration of the visibility of the figure. Further, the two acoustic lines illustrated in FIG. 2 correspond to two transducers 231 adjacent to each other in the scanning direction among the plurality of transducers 231.

The ultrasound diagnostic apparatus main body 1 visualizes an internal state of the subject as an ultrasound image, by using a reception signal from the ultrasound probe 2. As illustrated in FIG. 3, the ultrasound diagnostic apparatus main body 1 includes a transmission part 11, a reception part 12, a B-mode signal processing part 14, a display processing part 15, a display part 16, an operation input part 17, a controller 40, and the like.

The transmission part 11, the reception part 12, the B-mode signal processing part 14, and the display processing part 15 include, for example, at least one dedicated hardware (electronic circuit) corresponding to each process, such as a digital signal processor (DSP), an application specific integrated circuit (ASIC), or a programmable logic device (PLD).

The controller 40 includes a central processing unit (CPU) as a calculation and control device, a read only memory (ROM) and a random access memory (RAM) as a main memory, and the like. The ROM stores a basic program and basic setting data. The CPU reads a program corresponding to a processing content from the ROM, expands the program in the RAM, and executes the expanded program to centrally control an operation of each functional block (the transmission part 11, the reception part 12, the B-mode signal processing part 14, the display processing part 15, and the display part 16) of the ultrasound diagnostic apparatus main body 1.

In the present embodiment, a function of each functional block is implemented by cooperation between each hardware included in the functional block and the controller 40. Note that a part or all of the function of each functional block may be implemented by the controller 40 executing a program, or each functional block may have a configuration capable of executing the program.

The controller 40 includes a setting part 41 that sets a region of interest of an ultrasound image, and a scan controller 42 that sets a scanning method of the ultrasound probe 2. The region of interest is a partial region of the ultrasound image, and is, for example, a region that is a portion where an observer (user) desires detailed observation in a diagnosis region of the ultrasound diagnostic apparatus A.

Examples of the scanning method include a normal scanning method (hereinafter, normal scanning) and a trapezoid scanning method (hereinafter, trapezoid scanning). In the normal scanning and the trapezoid scanning, an acoustic line angle θ for transmission and reception is included in a scan condition.

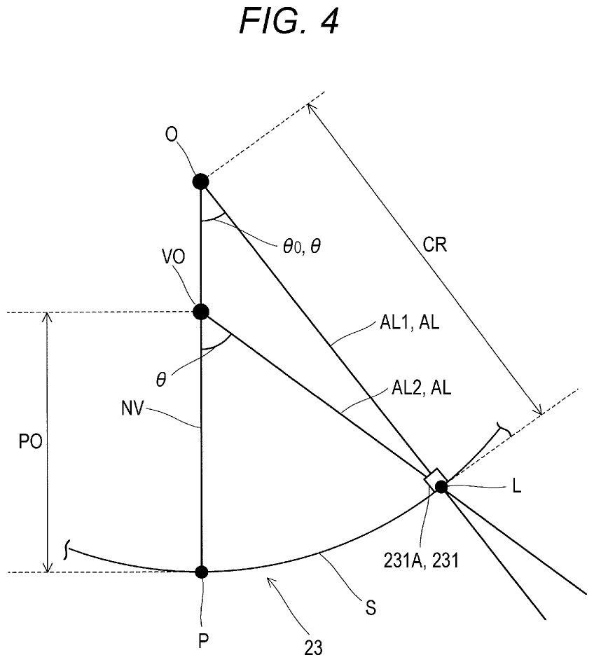

As illustrated in FIG. 4, the acoustic line angle θ is an angle formed by an acoustic line AL and a center normal line NV of the transducer surface S. The acoustic line AL is a center line of each beam of each transducer 231. The center normal line NV is a normal line passing through a probe origin O, which is a center of the arc of the transducer surfaces S, and through a center position P of the arc formed by the transducer surfaces S in a scanning direction.

The acoustic line angle θ is set for each beam emission point L of each transducer 231, and is expressed by an angle with a sign (±) based on the center normal line NV. Specifically, the acoustic line angle θ is represented with, for example, a sign + on a right side with respect to the center normal line NV, and a sign − on a left side with respect to the center normal line NV. In addition, the beam emission point L is a point at which the acoustic line AL of the ultrasound wave intersects with the transducer surface S.

The normal scanning is a scanning method (first scanning method) based on the probe origin O. In the normal scanning, a tangent line passing through the beam emission point L perpendicularly intersects with an acoustic line AL1 corresponding to the beam emission point L, and the acoustic lines AL1 of individual transducers 231 intersect at the probe origin O (first predetermined point). In addition, since the plurality of transducers 231 are arranged at equal intervals, inter-acoustic line angles AO of two adjacent acoustic lines AL are all equal. The inter-acoustic line angle Δθ is an angle formed by an acoustic line AL on an upstream side and an acoustic line AL on a downstream side in the scanning direction.

The trapezoid scanning is, for example, a scanning method based on a virtual origin VO on the center normal line NV. In the trapezoid scanning, for example, a point at which an acoustic line AL2 in a predetermined transducer 231A intersects with the center normal line NV is the virtual origin VO.

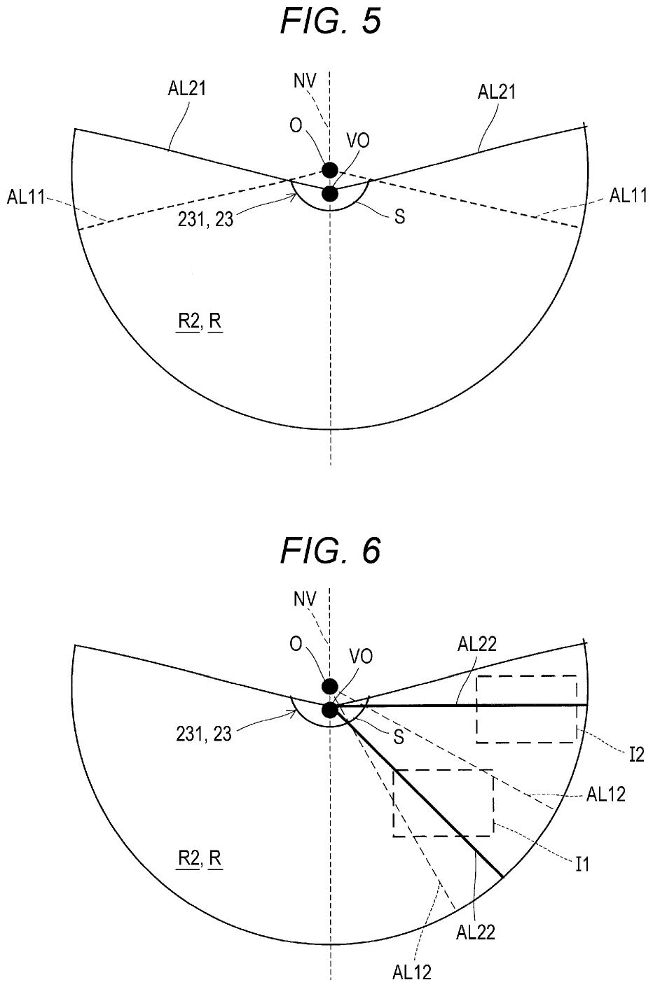

In the trapezoid scanning, since the virtual origin VO is shifted from the probe origin O, the inter-acoustic line angle Δθ is unlikely to be uniform between the individual acoustic lines. For example, in a case where the virtual origin VO is shifted to the transducer 231 side (− side) with respect to the probe origin O, the inter-acoustic line angle Δθ is more likely to spread than the normal scanning (see FIG. 5 and the like). Therefore, in the trapezoid scanning, it is possible to enlarge a diagnosis region as compared with the normal scanning. That is, the ultrasound diagnostic apparatus A can generate an ultrasound image related to a wide visual field range that is wider than a width of the transducer surface S of the ultrasound probe 2 in the scanning direction.

Here, a relationship of the following Equation (1) is established between the acoustic line angle θ and a position PO (hereinafter, a virtual origin position PO) of the virtual origin VO in the predetermined transducer 231A. The virtual origin position PO is represented by a distance between the virtual origin VO and the center position P of the transducer surface S.

$$θ = arctan [ C R \times sin {θ}_{0} / \{ P O - C R \times ( 1 - cos {θ}_{0} ) \} ] \qquad ( 1 )$$

CR is a length corresponding to a radius of the arc formed by the transducer surfaces S. θ₀ is an acoustic line angle in a case of the normal scanning in the predetermined transducer 231A. As the scan condition described above, the virtual origin position PO may be used instead of the acoustic line angle θ.

In addition, the virtual origin VO is any position on the center normal line NV or a position based on a function to be described later. Setting control of the scanning method in the scan controller 42 will be described later.

As illustrated in FIG. 3, in accordance with an instruction from the controller 40, the transmission part 11 generates a transmission signal (drive signal) and outputs the transmission signal to the ultrasound probe 2. Specifically, the transmission part 11 controls driving of the ultrasound probe 2 on the basis of the scanning method set by the controller 40. Although not illustrated, the transmission part 11 includes, for example, a clock generation circuit, a pulse generation circuit, a pulse width setting part, and a delay circuit.

The clock generation circuit generates a clock signal that determines a transmission timing and a transmission frequency of a pulse signal. The pulse generation circuit generates a bipolar rectangular wave pulse having a voltage amplitude that is set in advance at a predetermined period. The pulse width setting part sets a pulse width of a rectangular wave pulse outputted from the pulse generation circuit. The rectangular wave pulse generated by the pulse generation circuit is separated into wiring paths different for each transducer 231 of the ultrasound probe 2, before or after being inputted to the pulse width setting part. The delay circuit delays a generated rectangular wave pulse in accordance with a drive timing of each of the transducers 231 and outputs the rectangular wave pulse to the ultrasound probe 2.

By controlling the drive timing of the transducers 231, the acoustic line angles θ of a plurality of ultrasound waves transmitted in one time of scanning can be made different.

In accordance with an instruction from the controller 40, the reception part 12 receives a reception signal from the ultrasound probe 2 and outputs the reception signal to the B-mode signal processing part 14. Although not illustrated, the reception part 12 includes, for example, an amplifier, an A/D conversion circuit, and a phasing addition circuit.

The amplifier amplifies a reception signal corresponding to an ultrasound wave received by each transducer 231 of the ultrasound probe 2, at a predetermined amplification factor set in advance. The A/D conversion circuit converts the amplified reception signal into digital data at a predetermined sampling frequency. The phasing addition circuit applies a delay time to the A/D-converted reception signal for each wiring path corresponding to the transducer 231 to adjust a time phase, and adds the reception signal (phasing addition).

In accordance with an instruction from the controller 40, the B-mode signal processing part 14 generates B-mode image data by performing envelope detection processing, logarithmic compression processing, and the like on reception data for a B-mode image from the reception part 12, to adjust a dynamic range and a gain to perform luminance conversion.

In accordance with an instruction from the controller 40, the display processing part 15 converts image data generated by the B-mode signal processing part 14 into a display signal compatible with the display part 16 and outputs the display signal, and causes the display part 16 to display a B-mode image. Note that the display processing part 15 includes a digital scan converter (DSC) that performs coordinate conversion and pixel interpolation according to a type of the ultrasound probe 2.

When receiving a command to enlarge and display a region of interest from the operation input part 17 or the like, the display processing part 15 outputs a display signal to enlarge and display image data corresponding to the region of interest, to the display part 16.

The display part 16 includes, for example, a liquid crystal display, an organic EL display, a CRT display, or the like. In accordance with an instruction from the controller 40, the display part 16 displays an image on the basis of a display signal from the display processing part 15.

The operation input part 17 receives, for example, an input of information regarding diagnosis. The operation input part 17 includes, for example, an operation panel having a plurality of input switches, a keyboard, a mouse, and the like. The user can set a region of interest, a diagnosis site, a type of the ultrasound probe 2, and the like via the operation input part 17.

Note that an external device (for example, a tablet terminal) communicably connected to the ultrasound diagnostic apparatus main body 1 can also be applied to at least one of the display part 16 or the operation input part 17.

Next, setting control of a scanning method in the scan controller 42 will be described in detail.

The scan controller 42 sets a scanning method of the ultrasound probe 2 in accordance with a position of a region of interest (hereinafter, a region of interest position) set by the setting part 41. The setting of the region of interest by the setting part 41 is performed, for example, by the operation input part 17 being operated by the user.

Specifically, the scan controller 42 sets the scanning method in accordance with a region of interest position set in a case where an ultrasound image related to a wide visual field range is generated. In a case where the region of interest is set in a scannable range of the ultrasound probe 2, the scan controller 42 sets the scanning method after setting of the region of interest such that a density of acoustic lines in the region of interest is higher than that before setting of the region of interest.

The scannable range is a range that is the maximum range of the diagnosable region of the ultrasound diagnostic apparatus A. As illustrated in FIG. 5, in the trapezoid scanning, the virtual origin VO may be located on the − side (lower side) with respect to the probe origin O of the normal scanning. In this case, an acoustic line AL21 to the transducer 231 at an end in the scanning direction is positioned above an acoustic line AL11 to the transducer 231 at an end in the normal scanning That is, in the trapezoid scanning, a fan shape (R2 portion) of the diagnosis region R can be enlarged more than a fan shape (R1 portion in FIG. 2) of the diagnosis region of the normal scanning.

There are two types of trapezoid scanning methods, namely, a method with a fixed virtual origin point (hereinafter, fixed) and a method with a mobile virtual origin point (hereinafter, mobile).

The fixed trapezoid scanning (a second scanning method) is a scanning method different from the normal scanning method, and is a scanning method in which a position of the virtual origin VO is fixed in one time of scanning. In the fixed trapezoid scanning, the virtual origin VO is set at a position different from a position of the probe origin O (a center of the arc of the transducer surfaces S) of the normal scanning, and individual acoustic lines intersect at the virtual origin VO.

The mobile trapezoid scanning (a third scanning method) is a scanning method different from the normal scanning method, and is a scanning method in which a position of the virtual origin VO moves in one time of scanning. In the mobile trapezoid scanning, the virtual origin position PO varies for each transducer 231. That is, in the mobile trapezoid scanning, the center normal line NV and each acoustic line intersect at a plurality of points.

The acoustic line angle θ in the mobile trapezoid scanning is expressed by the following functions of Equations (2) and (3).

$$θ = s g n ( L ) \times β \times {\vert CR \times sin {θ}_{0} \vert}^{α} \qquad ( 2 )$$

$$PO = ( CR \times sin {θ}_{0} / tan \{ sgn ( L ) \times β \times {\vert CR \times sin {θ}_{0} \vert}^{α} \} + CR \times ( 1 - cos {θ}_{0} ) \qquad ( 3 )$$

sgn (L): sign of beam emission point

α, β: deflection angle variation coefficient

Here, the deflection angle variation coefficients α and β are characteristic values for controlling the inter-acoustic line angle Δθ, and have a relationship in which β is obtained when α is a constant and the beam emission point L of the transducer 231 located at the most downstream end in the scanning direction and the acoustic line angle θ corresponding to the beam emission point L are defined.

By using the trapezoid scanning as described above, the scannable range of the ultrasound diagnostic apparatus A can be expanded up to a second diagnosis region R2 wider than a first diagnosis region R1 (see FIG. 2) of the normal scanning, and accordingly, it is possible to generate an ultrasound image related to a wide visual field range.

In addition, as illustrated in FIG. 6, the inter-acoustic line angle Δθ in the trapezoid scanning tends to be wider than the inter-acoustic line angle Δθ in the normal scanning A region surrounded by AL12 in FIG. 6 is a region sandwiched by the acoustic lines related to the two adjacent transducers 231 in the normal scanning, and a region surrounded by AL22 is a region sandwiched by the acoustic lines related to the transducers 231 in the trapezoid scanning.

As described above, in the trapezoid scanning in which the inter-acoustic line angle Δθ becomes wide, a density of acoustic lines in the diagnosis region is lower than that in the normal scanning Therefore, image quality of the image data in the diagnosis region in the trapezoid scanning becomes worse than image data in the diagnosis region in the normal scanning.

The scan controller 42 changes a method of setting the inter-acoustic line angle Δθ in accordance with a region of interest position. Specifically, the scan controller 42 selects either the normal scanning or the trapezoid scanning in accordance with the region of interest position.

Here, it is assumed that the ultrasound diagnostic apparatus A can apply two scanning methods of the normal scanning and the fixed trapezoid scanning that is based on the virtual origin VO located on the − side of the probe origin O.

It is assumed that a region of interest is set in the second diagnosis region R2 when diagnosis is performed in the second diagnosis region R2 wider than the first diagnosis region R1 by applying the trapezoid scanning, by using the ultrasound diagnostic apparatus A.

For example, in a case where a region of interest position is set in a portion (a region of I1) including the first diagnosis region R1 in the second diagnosis region R2, it is possible to apply two scanning methods, the normal scanning and the trapezoid scanning, in the region of interest position in this region.

In a case where two or more scanning methods are applicable at the set region of interest position, the scan controller 42 selects a scanning method having the highest acoustic line density.

In the case of the trapezoid scanning in which a diagnosis region is enlarged, each inter-acoustic line angle Δθ tends to be wider than the inter-acoustic line angle Δθ of the normal scanning Therefore, when the region of interest is located in the first diagnosis region R1, the acoustic line density in the region of interest I1 is higher when the normal scanning is applied. Therefore, the scan controller 42 sets the normal scanning as the scanning method.

Further, when the region of interest position is set to an end position (a region of I2) in the second diagnosis region R2, the trapezoid scanning method alone can be applied since the region of interest position in this region is out of the range of the normal scanning. Therefore, the scan controller 42 sets the trapezoid scanning as the scanning method.

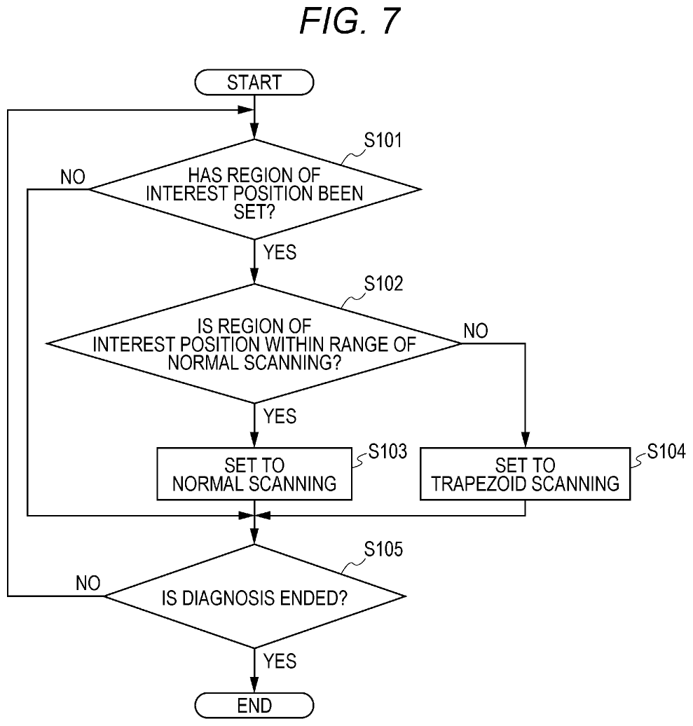

Next, an operation example when setting control of the scanning method is executed by the controller 40 will be described. FIG. 7 is a flowchart illustrating an example of an operation example of setting control of the scanning method in the controller 40. The processing in FIG. 7 is appropriately executed, for example, while the ultrasound diagnostic apparatus A executes diagnosis with the trapezoid scanning.

As illustrated in FIG. 7, the controller 40 determines whether or not a region of interest position has been set (step S101). As a result of the determination, when the region of interest position has not been set (step S101, NO), the process proceeds to step S105.

Whereas, when the region of interest position has been set (YES in step S101), the controller 40 determines whether or not the region of interest position is within a range of the normal scanning (step S102).

As a result of the determination, when the region of interest position is within the range of the normal scanning (YES in step S102), the controller 40 sets the scanning method to the normal scanning (step S103). Whereas, when the region of interest position is not within the range of the normal scanning (step S102, NO), the controller 40 sets the scanning method to the trapezoid scanning (step S104).

After step S103 or step S104, the controller 40 determines whether or not diagnosis in the ultrasound diagnostic apparatus A has ended (step S105). As a result of the determination, when the diagnosis has not ended (step S105, NO), the process returns to step S101. Whereas, when the diagnosis has ended (YES in step S105), this control is ended.

According to the present embodiment configured as described above, since the scanning method is set in accordance with a region of interest position, the region of interest position can be scanned by an appropriate scanning method.

Specifically, in a case where the controller 40 sets the region of interest position at a position closer to the center than the end position in the scanning direction, the image quality of the region of interest close to the center can be improved since the scanning method is set so as to increase the density of acoustic lines in the region of interest. In addition, at an end position in the scanning direction, the scanning method (the trapezoid scanning) corresponding to a range of the end position is set, so that image data of the region of interest can be reliably secured.

That is, in the present embodiment, it is possible to handle various region of interest positions while improving the image quality of the region of interest.

In the above-described embodiment, the fixed trapezoid scanning is exemplified, but the present invention is not limited thereto, and the mobile trapezoid scanning may be applied. In a case of the mobile trapezoid scanning, for example, a range of the diagnosis region may be set by appropriately setting a value of the deflection angle variation coefficient β.

Further, in the mobile trapezoid scanning, the value of the deflection angle variation coefficient β may be a fixed value or a variable value.

The scanning method is to be the normal scanning when the region of interest position is set in the region (the first diagnosis region R1) that can be diagnosed by the normal scanning, while the scanning method is to be the trapezoid scanning when the region of interest position is set at a position including the second diagnosis region R2. Between the normal scanning and the trapezoid scanning, a density of acoustic lines is different. Therefore, when the user observes image data of a region of interest generated by the trapezoid scanning after observing image data of a region of interest generated by the normal scanning, a variation range of the image quality of both image data may be conspicuous, for example, since the inter-acoustic line angle Δθ is larger in a case of the trapezoid scanning than in a case of the normal scanning.

That is, when a value of the deflection angle variation coefficient β is a fixed value, there is a possibility that image quality related to the region of interest greatly changes depending on whether or not the region of interest position crosses a boundary between the first diagnosis region R1 and the second diagnosis region R2 (a line corresponding to the acoustic line AL11 in FIG. 5).

Therefore, the scan controller 42 changes the deflection angle variation coefficient β (a parameter for determining the inter-acoustic line angle Δθ) in accordance with the region of interest position. Specifically, the scan controller 42 determines a value of the deflection angle variation coefficient β in accordance with the region of interest position such that the value of the deflection angle variation coefficient β gradually increases toward the end position in the scanning direction.

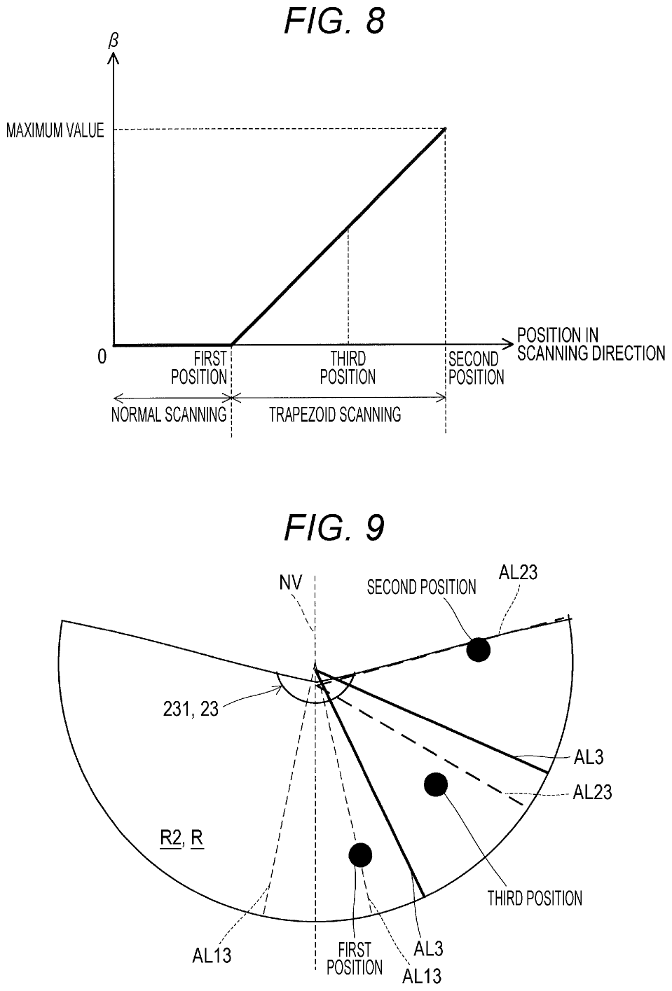

For example, as illustrated in FIG. 8, the scan controller 42 determines the value of the deflection angle variation coefficient β in accordance with a function in which the value of the deflection angle variation coefficient β increases linearly between a first position and a second position in the diagnosis region R. In FIG. 8, a vertical axis represents a value of the deflection angle variation coefficient β, and a horizontal axis represents a position in the scanning direction in the diagnosis region R. In FIG. 8, 0 indicates a position of the center normal line NV, the first position indicates the most downstream position where the normal scanning is set as the scanning method, for example, and the second position indicates an end position of the diagnosis region R.

Note that FIG. 8 illustrates a function corresponding to a position on the + side with respect to the center normal line NV, and a function corresponding to a position on the − side with respect to the center normal line NV is obtained by making the function corresponding to the position on the + side symmetrical with respect to the center normal line NV.

Then, the scan controller 42 sets the scanning method to the trapezoid scanning according to a determined value of the deflection angle variation coefficient β.

Thus, as illustrated in FIG. 9, when a region on the upstream side of the first position is set as the region of interest position in the diagnosis region R, the normal scanning (the value of the deflection angle variation coefficient β is 0) is set as the scanning method. The inter-acoustic line angle Δθ of the normal scanning corresponds to, for example, a range surrounded by an acoustic line AL13.

When a region including the second position is set as the region of interest position in the diagnosis region R, the trapezoid scanning in which the value of the deflection angle variation coefficient β is determined to be the maximum value in FIG. 8 is set as the scanning method. In this case, the inter-acoustic line angle Δθ of the trapezoid scanning corresponds to, for example, a range surrounded by an acoustic line AL23.

Further, in the diagnosis region R, when a region including a third position that is an intermediate position between the first position and the second position is set as the region of interest position, the trapezoid scanning in which the value of the deflection angle variation coefficient β is determined to be a value corresponding to the third position in FIG. 8 is set as the scanning method. In this case, the inter-acoustic line angle Δθ of the trapezoid scanning corresponds to, for example, a range surrounded by an acoustic line AL3. This range is larger than the range surrounded by the acoustic line AL13 and smaller than the range surrounded by the acoustic line AL23.

That is, the inter-acoustic line angle Δθ of the trapezoid scanning at the third position is a value between an inter-acoustic line angle related to the first position and an inter-acoustic line angle related to the second position. As a result, even when the observer observes both the region of interest on the upstream side and the region of interest on the downstream side of a boundary where the scanning method is switched, a variation range of the image quality of both the regions of interest can be reduced.

Further, in the above-described embodiment, the normal scanning is included in the selectable scanning methods, but the present invention is not limited thereto, and the normal scanning may not be included in the selectable scanning methods.

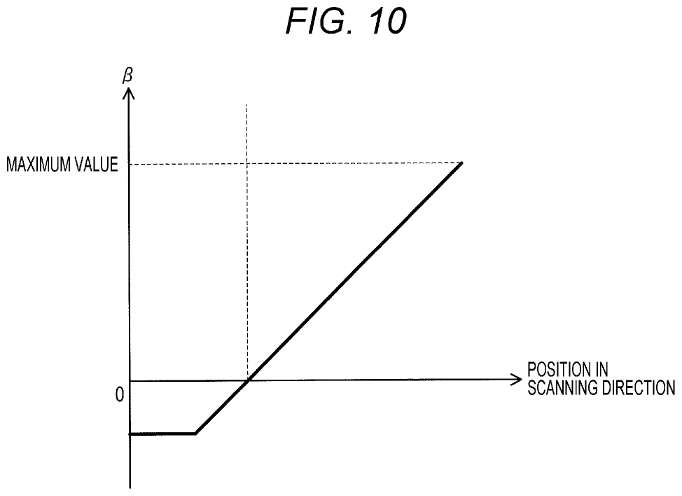

In this case, as illustrated in FIG. 10, similarly to FIG. 8, the scan controller 42 sets the scanning method so as to change the deflection angle variation coefficient β in the mobile trapezoid scanning in accordance with the region of interest position. In FIG. 10, the value of the deflection angle variation coefficient β is set to a negative value in a range of the normal scanning in FIG. 8.

When the value of the deflection angle variation coefficient β is set to a negative value, a position of the virtual origin VO becomes a position on the + side of the probe origin O. Therefore, the inter-acoustic line angle Δθ is narrowed, and the density of acoustic lines in the diagnosis region increases.

Therefore, for example, when the region of interest position is set in a range including a center portion in the scanning direction in the diagnosis region, the deflection angle variation coefficient is set to a negative value, and the deflection angle variation coefficient increases toward the end position and is set to a value reaching the maximum value of the deflection angle variation coefficient at the end position.

In this way, since the density of acoustic lines is higher in the center portion of the diagnosis region than in the end portion, image quality of image data of a region of interest near the center portion can be improved when the region of interest position is set near the center portion.

Further, in addition to changing the deflection angle variation coefficient (a method of setting an inter-acoustic line angle) in accordance with the region of interest position in the mobile trapezoid scanning, a position of the virtual origin VO may be changed in accordance with the region of interest position in the fixed trapezoid scanning.

Further, in the above-described embodiment, the mobile trapezoid scanning and the fixed trapezoid scanning are handled separately, but the present invention is not limited thereto, and options of the scanning method may include an option of the mobile trapezoid scanning and the fixed trapezoid scanning.

For example, it is generally known that, in the fixed trapezoid scanning, when a position of the virtual origin VO is located on the − side of the probe origin O, an inter-acoustic line angle is smaller on an end side in a scanning direction than on a center side, as described in JP No. 2020-130736 A.

Then, it is assumed that the mobile trapezoid scanning in which the inter-acoustic line angle is set to be small on the center side in the scanning direction is applied in the ultrasound diagnostic apparatus A, as in the technique described in JP 2020-130736 A, for example.

In such a case, the scan controller 42 sets the mobile trapezoid scanning when the region of interest position is set on the center side in the scanning direction, and sets the fixed trapezoid scanning is set when the region of interest position is set on the end side in the scanning direction. That is, the scan controller 42 selects either the mobile trapezoid scanning or the fixed trapezoid scanning in accordance with the region of interest position.

In this way, it is possible to improve the image quality of the region of interest while widening the diagnosis region.

In addition, the scan controller 42 may have a configuration of being able to select any one of the normal scanning, the fixed trapezoid scanning, and the mobile trapezoid scanning.

Furthermore, in the above-described embodiment, the scanning method is set in accordance with the region of interest position set in a case where an ultrasound image related to a wide visual field range is generated, but the present invention is not limited thereto. For example, the scanning method may be set in accordance with a region of interest position set in a case where an ultrasound image related to a normal range (a scanning range of the normal scanning) is generated.

In this case, options of the scanning method desirably include the trapezoid scanning in which, in a center portion of the diagnosis region, a position of a virtual origin is set on the + side of the position of the probe origin and an inter-acoustic line angle is narrowed.

Further, in the above-described embodiment, the mobile trapezoid scanning is a method related to the functions of Equations (2) and (3), but the present invention is not limited thereto, and the mobile trapezoid scanning may be a method related to a function other than the functions of Equations (2) and (3).

Further, in the above-described embodiment, the ultrasound probe having the arc-shaped transducer surface has been exemplified, but the present invention is not limited thereto, and the ultrasound probe may have a transducer surface other than the arc-shaped transducer surface, such as a linear transducer surface. For example, when a linear probe is used as the ultrasound probe, a method related to a function described in JP 2020-130736 A is to be the mobile trapezoid scanning.

In addition, the above-described embodiment is merely an example of implementation in carrying out the present invention, and the technical scope of the present invention should not be construed in a limited manner by these. That is, the present invention can be implemented in various forms without departing from the scope or main features of the present invention.

Although embodiments of the present invention have been described and illustrated in detail, the disclosed embodiments are made for purposes of illustration and example only and not limitation. The scope of the present invention should be interpreted by terms of the appended claims

What is claimed is:

1. An ultrasound diagnostic apparatus for generating an ultrasound image of a subject by driving an ultrasound probe in which a plurality of transducers are arranged in an array, the ultrasound diagnostic apparatus comprising a hardware processor that
sets a region of interest of the ultrasound image, and
sets a scanning method of the ultrasound probe in accordance with a position of the set region of interest.

2. The ultrasound diagnostic apparatus according to claim 1, wherein
in a case where the region of interest is set in a scannable range of the ultrasound probe, the hardware processor sets the scanning method after setting of the region of interest to increase a density of acoustic lines in the region of interest as compared with a density of acoustic lines before setting of the region of interest.

3. The ultrasound diagnostic apparatus according to claim 1, wherein
the hardware processor changes a method of setting an inter-acoustic line angle of two adjacent acoustic lines among acoustic lines related to the plurality of transducers, in accordance with a position of the region of interest.

4. The ultrasound diagnostic apparatus according to claim 3, wherein
the hardware processor is capable of setting:
a first scanning method in which a tangent line passing through a beam emission point perpendicularly intersects with an acoustic line corresponding to the beam emission point, the beam emission point being at an intersection of each of the acoustic lines and a surface of each of the transducers; and
a second scanning method that is a scanning method different from the first scanning method and in which the acoustic lines each intersect at a predetermined point, and
any one of the first scanning method or the second scanning method is selected in accordance with a position of the region of interest.

5. The ultrasound diagnostic apparatus according to claim 3, wherein
the hardware processor is capable of setting:
a first scanning method in which a tangent line passing through a beam emission point perpendicularly intersects with an acoustic line corresponding to the beam emission point, the beam emission point being at an intersection of each of the acoustic lines and a surface of each of the transducers; and
a third scanning method in which a plurality of points where a normal line intersects with each of the acoustic lines are set, the normal line passing through a center of a transducer surface of the ultrasound probe in a scanning direction, and
any one of the first scanning method or the third scanning method is selected in accordance with a position of the region of interest.

6. The ultrasound diagnostic apparatus according to claim 3, wherein
the hardware processor is capable of setting
a third scanning method in which a plurality of points where a normal line intersects with each of the acoustic lines are set, the normal line passing through a center of a transducer surface of the ultrasound probe in a scanning direction, and
a parameter to determine the inter-acoustic line angle is changed in accordance with a position of the region of interest.

7. The ultrasound diagnostic apparatus according to claim 3, wherein
the hardware processor is capable of setting at least two of:
a first scanning method in which a tangent line passing through a beam emission point perpendicularly intersects with an acoustic line corresponding to the beam emission point, the beam emission point being at an intersection of each of the acoustic lines and a surface of each of the transducers;
a second scanning method that is a scanning method different from the first scanning method and in which the acoustic lines each intersect at a predetermined point; or
a third scanning method in which a plurality of points where a normal line intersects with each of the acoustic lines are set, the normal line passing through a center of a transducer surface of the ultrasound probe in a scanning direction, and
any of the at least two is selected in accordance with a position of the region of interest.

8. The ultrasound diagnostic apparatus according to claim 1, wherein
the ultrasound diagnostic apparatus is capable of generating an ultrasound image related to a wide visual field range that is wider than a width of a transducer surface of the ultrasound probe in a scanning direction, and
the hardware processor sets the scanning method in accordance with a position of a region of interest set in a case where the ultrasound image related to the wide visual field range is generated.

9. The ultrasound diagnostic apparatus according to claim 1, further comprising:
a display processing part that outputs a signal to enlarge and display an inside of a region of interest of an ultrasound image; and
a display part that displays the ultrasound image based on a signal of the display processing part.

10. A setting control method of an ultrasound diagnostic apparatus for generating an ultrasound image of a subject by driving an ultrasound probe in which a plurality of transducers are arranged in an array, the setting control method comprising:
setting a region of interest of the ultrasound image; and
setting a scanning method of the ultrasound probe in accordance with a position of the set region of interest.

11. A non-transitory recording medium storing a computer readable setting control program of an ultrasound diagnostic apparatus for generating an ultrasound image of a subject by driving an ultrasound probe in which a plurality of transducers are arranged in an array, the setting control program causing a computer to execute:
setting a region of interest of the ultrasound image; and
setting a scanning method of the ultrasound probe in accordance with a position of the set region of interest.
